(19) United States
(12) Patent Application Publication
(10) Pub. No.: US 2017/0296694 A1
(43) Pub. Date: Oct. 19, 2017

(54) BIOLOGICAL GLUE AND USE THEREOF AS A MEDICAMENT

(71) Applicant: LABORATOIRE FRANCAIS DU FRACTIONNEMENT ET DES BIOTECHNOLOGIES, Les Ulis (FR)

(72) Inventors: Abdessatar Sami CHTOUROU, ELANCOURT (FR); Jean-Luc PLANTIER, CROIX (FR)

(73) Assignee: Laboratoire Francais du Fractionnement et des Biotechnologies, Les Ulis (FR)

(21) Appl. No.: 15/517,038

(22) PCT Filed: Oct. 7, 2015
(86) PCT No.: PCT/EP2015/073169

§ 371 Date: Apr. 5, 2017

(30) Foreign Application Priority Data
Oct. 7, 2014 (FR) 14 59614

(51) Int. Cl. A61L 24/00; A61L 26/00; A61L 24/10

(52) U.S. Cl. CPC A61L 24/0015; A61L 26/0047; A61L 24/108; A61L 26/0038; A61L 24/106; A61L 26/0042; A61L 2300/252; A61L 2300/418; A61L 2400/04

(57) ABSTRACT

Disclosed is a thrombin-free, liquid biological glue for therapeutic use, including fibrinogen and factor VIIa. The ratio of fibrinogen concentration to FVIIa concentration is 20000:1 to 1000:1, with the concentrations being expressed in weight per volume. The fibrinogen concentration is lower than 60 mg/ml. Also disclosed are a kit for preparing such a biological glue, a method to prepare the glue, and a medicament.

The present invention relates to a biological glue containing fibrinogen and activated Factor VII. The present invention also relates to a biological glue containing fibrinogen, activated factor VII and a source of calcium ions.

By <<biological glue>> is meant a component capable of joining tissue elements (skin, bone, various organs) and at the same time of ensuring haemostasis of the damaged tissues, thereby being able to strengthen, complete or replace the joining of tissues by one or more sutures. Said component also allows enhanced repair/healing, in particular by providing possible slow diffusion of active compounds.

Blood coagulation occurs via a cascade of steps involving different proenzymes and procofactors contained in blood which are converted via proteolytic enzymes to their activated form. This succession of steps, or coagulation cascade, takes place via two coagulation systems known as the extrinsic coagulation pathway and intrinsic coagulation pathway, leading to conversion of prothrombin to thrombin.

The extrinsic pathway involves the action of Factor VII contained in blood. However, this factor requires activation resulting in activated factor VII or factor VIIa to initiate this coagulation cascade. Factor VIIa has low enzymatic activity until it complexes to the tissue factor of phospholipid type released after tissue damage. Factor VIIa thus complexed converts factor X (FX) to factor Xa (FXa) in the presence of calcium ions. Factor Xa in turn converts prothrombin to thrombin which activates factor V (Factor Va). Thrombin also activates factor XIII to factor XIIIa. Thrombin, in the presence of calcium, acts on fibrinogen converting it to fibrin. The presence of factor XIIIa allows the formation of a fibrin clot with solid, adherent meshed latticework which is progressively and slowly resorbed with the installation of consolidating healing tissue for which the latticework acts as matrix.

The intrinsic coagulation pathway comprises a cascade of reactions leading to activation of thrombin via factor XII (FXII). The latter activates factor XI (Factor XIa-FXIa) which activates factor IX (Factor IXa-FIXa) in the presence of phospholipid tissue factors (TF). Factor IXa takes part in activating Factor X to Factor Xa in the presence of factor VIIIa, tissue factor and calcium ions. This then leads to the conversion of prothrombin to thrombin. It is to be noted that the presence of Factor Xa or of thrombin also allows activation of factor VII (Factor VIIa).

It is therefore apparent that factor VIIa plays a predominant role in the mechanisms of intrinsic coagulation, leading to the formation of a blood clot. It is used in the treatment of haemophilia A patients having a circulating inhibitor i.e. a specific antibody which limits or prevents activation of factor VIII (FVIII). Factor VIIa has the advantage of being able to act locally in the presence of tissue factor released after tissue damage generating haemorrhages, even in the absence of factor VIII or IX.

However, in the event of haemorrhagic tissue injury generated by cuts, wounds or external surgical procedure, tissue repair by suture is a necessity in particular to promote the arresting of haemorrhage through the formation of a fibrin clot, in accordance with the mechanisms described above. It may be necessary, in particular in the event of heavy surgical procedure, to promote haemostasis of the damaged tissues.

Aside from haemostasis of damaged tissue, it is also required to accelerate, reinforce even complete the natural healing process of tissues, with or without suture, through the joining or tissues to prevent their dehiscence. This is particularly possible with the local application of biological glue. Biological glues are formed of a mixture of blood coagulation circulating factors, in particular fibrinogen and Factor XIII. They also require the exogenous adjunct of thrombin, an enzyme needed to convert fibrinogen to insoluble fibrin, cross-linkable by factor XIII. Additional components are needed to obtain coagulation of fibrinogen, in particular calcium ions and optionally aprotinin that is added for its anti-fibrinolytic properties.

Biological glues can also be used to secure biomaterials of various types (collagen, alginates and polylactic acid) to biological tissues to reinforce the mechanical properties thereof until consolidation occurs through the natural regrowth of body cells e.g. for artificial skin.

Biological glues can also be used as a reservoir for the dispensing over time, as and when they are absorbed, of different active compounds such as antibiotics, growth or cell stimulation factors.

Commercially available biological glues are in kit form comprising at least the four above-mentioned components in dried form in which the thrombin-activated proteins, fibrinogen and Factor VIII, must be isolated from the thrombin since the association thereof generates polymerisation to fibrin within a very short time, in the order of a few seconds after reconstitution in a liquid and mixing. This is why biological glue kits are at least bi-component i.e. comprising a pack containing fibrinogen and Factor XIII and another pack containing thrombin. The biological glue fulfils the above-indicated functions through the reconstituting and mixing of the two packs, e.g. using syringes and needles, followed by application to the tissue to be sealed or by spraying onto the wound.

However, such reconstitution and mixing is relatively complex and the source of possible error, in particular in an emergency, since as soon as the fibrinogen and thrombin are contacted in liquid form, in the presence of the other components, fibrin is formed almost instantly. Fibrinogen-thrombin mixing can therefore only be carried out just before application to the wound or to the operative site, or directly onto the wound. In addition, if a single biological glue dispensing device is used, there is a true risk of fibrin setting in the device itself which may lead to clogging of the dispensing and application system. Also, since the product is not stable, it may be difficult to plan the required volume before the start of procedure. Emergency reconstitution of the lyophilised product is accompanied by a waiting time for the product to stabilise, which may be detrimental to the proper conducting of a surgical procedure.

The above-mentioned shortcomings therefore raise problems, in particular with respect to surgical procedures and/or emergency situations when the rapidity of care is a necessity. It is observed that such disadvantages are likely to generate non-homogeneous joining of tissues through the formation of fibrin clusters before tissue adhesion, which may further be accentuated by shrinkage of the clot finally leading to irregular scarring lending itself to dehiscence and secondary post-operative bleeding.

To overcome these shortcomings, the Applicant has developed a biological glue meeting several joint objectives. The first objective is to provide a biological glue comprising coagulation factors free of thrombin, without the risk of fibrin formation before application to a biological tissue and capable of being stored in the form of a homogeneous solute. The second objective is to provide a glue also having increased capacity for haemostasis of damaged tissues and for joining of tissues leading to improved healing.

Application WO2007080276 describes a single-compound liquid biological glue free of thrombin and comprising fibrinogen, factor VIIa and a source of calcium ions.

However, the efficacy of this biological glue may be limited in the event of major bleeding when the blood flow tends to disperse the biological glue and wash the glue away from the site of injury or away from the damaged tissue.

The Applicant has developed an improved formulation of the described biological glue by optimising the concentration of the components, fibrinogen and factor VII, in the glue. The new formulation is more effective for the haemostasis of damaged tissue and allows improved healing whilst having a lower production cost. In addition, the new biological glue of the invention advantageously has increased adhesive properties.

The Applicant has also researched this formulation to include in the composition at least one gelling agent which allows a reduction in or the avoidance of risks of dispersion of the biological glue outside the site of injury in the event of bleeding, especially major bleeding. The Applicant has also shown that the presence of the gelling agent further increases the stability of the biological glue at ambient temperature, compared with a formulation not containing a gelling agent.

Finally, the Applicant has shown that a reduced concentration of fibrinogen, in particular lower than 60 mg/ml for FVIIa concentrations of 0.34 to 34 UI/ml, is advantageous for the biological glue to have improved adhesive power and haemostasis properties. There is therefore an optimal ratio of fibrinogen concentration to FVIIa concentration (the concentrations being expressed in weight per volume) in the biological glue, this ratio being 60000:1 to 1000:1.

### Biological Glue

The invention therefore concerns a thrombin-free, liquid biological glue for therapeutic use, comprising fibrinogen and Factor VIIa wherein the ratio of fibrinogen concentration to FVIIa concentration (the concentrations being expressed in weight per volume) is 60000:1 to 1000:1. According to some embodiments, the biological glue further comprises a source of calcium ions and/or at least one gelling agent.

The invention also concerns a thrombin-free, liquid biological glue for therapeutic use comprising fibrinogen, Factor VIIa and at least one gelling agent. According to some embodiments, the biological glue further comprises a source of calcium ions.

The biological glue is in the form of a pharmaceutical composition i.e. a <<single-compound>> formulation in which the constituent glue components of interest form a single liquid or lyophilised composition to be reconstituted, as opposed to the bi-compound glues in the prior art.

In the invention, any prior art fibrinogen (Fbg) and activated Factor VII (FVIIa), preferably recombinant, are suitable for producing the glue, provided however that they are compatible with the calcium ions which may be added to the glue obtained in liquid form i.e. their contacting does not generate the conversion of fibrinogen to fibrin, in particular for at least 24 hours before use. The two active ingredients, fibrinogen and FVIIa, must therefore be free of residual thrombin (FIIa) but also free of coagulation factors which, in the presence of calcium ions, would trigger the cascade of intrinsic or extrinsic coagulation. Such coagulation factors are factor II (FII) and factor X (FX), and preferably the prothrombin factors (Factors II, IX and X) and in particular in their activated form.

For example, a maximum acceptable content of FII and FX in the fibrinogen is about 0.1 UI/g fibrinogen for each one thereof.

Preferably, the fibrinogen and FVIIa of the glue are not derived from pre-activated plasma.

The fibrinogen, preferably made virally safe, can be prepared using any partial or complete plasma fractionating technique known in the prior art. It may be the technique described in EP 0 305 243 or the technique developed by the Applicant in patent application FR 05 06640 wherein a fibrinogen concentrate can be obtained. It is also possible to use recombinant fibrinogen from cell line production or from transgenic animals in particular in the milk thereof. It may also be transgenic fibrinogen produced and purified in accordance with the method described in patent applications WO95/23868, WO00/17234, WO00/17239 or WO2009/134130, in particular as produced in the milk of transgenic bovines.

The term “fibrinogen” comprises all natural allelic variations of fibrinogen which may exist and any form or degree of glycosylation or any other post-translational modification. Fibrinogen is naturally subjected to phosphorylation, sulfation and glycosylation. The term “fibrinogen” also comprises variants of fibrinogen having similar or higher biological activity compared with the activity of the wild type, these variants particularly including polypeptides differing from the wild type fibrinogen through insertion deletion or substitution of one or more amino acids.

The preparation of factor VIIa, particularly in the form of a concentrate, preferably made virally safe, is also known. For example, mention can be made of patent EP 346 241. It is also possible to use recombinant or transgenic factor VIIa, in particular as described in document FR 0604872. According to one preferred embodiment, human factor VIIa is produced in the milk of non-human transgenic mammals, genetically modified to produce this protein. Preferably, it is produced in the milk of a transgenic rabbit or goat. Secretion of factor VIIa by the mammary glands, allowing secretion thereof in the milk of a transgenic mammal, implies tissue-dependent control over the expression of factor VIIa. Such control methods are well known in the state of the art. Expression control is obtained by using sequences allowing expression of the protein in a particular tissue of the animal. These sequences comprise WAP promoter, beta-casein, beta-lactoglobulin sequences and peptide signal sequences. In particular, a method to extract proteins of interest from the milk of transgenic animals is described in patent EP 0 264 166.

In particular, the factor VIIa used in the invention may be human factor VIIa produced in the milk of a transgenic rabbit such as described in Chevreux et al, Glycobiology 2013 December; 23 (12): 1531-46.

Alternatively, the factor VIIa can be produced by genetic engineering from BHK baby hamster kidney cells. For example, the factor VIIa may be NovoSeven® Factor VIIa, authorised on the European market since 1996 and on the American market in 1999, produced by the Danish company Novo Nordisk. The factor VIIa may also be a NovoSeven variant called NovoSeven RT®. The term <<factor VIIa>> or <<activated factor VII>> also comprises variants of Factor VII which have similar or higher biological activity than the wild type, these particular variants including polypeptides differing from the wild type of factor VIIa through insertion, deletion or substitution of one or more amino acids.

According to one embodiment, the glue of the invention also comprises factor XIII. An exogenous addition of factor XIII promotes cross-linking of the fibrin network, and hence its coagulating and healing power. Preferably, the biological glue comprises FXIII when the fibrinogen is recombinant. The Applicant has observed that the biological glue including recombinant fibrinogen leads to a reduced rate of coagulation, and reduced amplitude and firmness of the clots formed, and this reduction may be partly or wholly offset through the addition of FXIII to the biological glue.

According to one embodiment, the glue of the invention further comprises a source of calcium ions and factor XIII.

The factor XIII, preferably virally safe, can be isolated from plasma using any method developed in the prior art, and may advantageously form the accompanying protein of fibrinogen at the time of plasma fractionating. In this case, it is preferred to use the method described in patent application FR 05 06640. It is also possible to use recombinant factor VIII from mammal or yeast cell line production, or transgenic produced in the milk of transgenic animals. This active ingredient must meet the same purity criteria however as set forth above, in particular with regard to the presence of some other plasma factors if the factor VIII is of plasmatic origin.

According to another embodiment, the biological glue does not contain any coagulation factor other than fibrinogen and FVIIa. Said glue has the advantage of only requiring two coagulation factors which limits preparation costs of the glue on an industrial scale due to the presence of a minimum but efficient number of active components.

According to another embodiment, the biological glue comprises a source of calcium ions and does not contain any coagulation factor other than fibrinogen and FVIIa.

According to one embodiment, the glue of the invention further comprises a source of calcium ions. The sources of calcium ions are water-soluble components compatible with clinical use. Preferably these components are inorganic salts, such as calcium chloride (CaCl₂) or calcium gluconate.

The constituent components of the glue are contained in efficient amounts allowing the glue to meet the desired therapeutic objectives.

Therefore, the biological glue advantageously has a fibrinogen content of less than 60 mg per ml. In particular, the biological glue has a fibrinogen content lower than 55 mg per ml, lower than 50 mg per ml, lower than 45 mg per ml, lower than 40 mg per ml, lower than 35 mg per ml, lower than 30 mg per ml, lower than 25 mg per ml, lower than 20 mg per ml, lower than 15 mg per ml, lower than 10 mg per ml or lower than 5 mg per ml. In particular, the biological glue has a fibrinogen content of 0.1 mg to 60 mg per ml of biological glue, preferably 0.1 to 45 mg/ml, more preferably 0.1 to 40 mg/ml, 0.2 to 60 mg/ml, 0.3 to 40 mg/ml, 0.5 to 30 mg/ml, 1 to 20 mg/ml or even 1 to 10 mg/ml.

The biological glue has a FVIIa content of 0.1 μg to 10 μg per ml of biological glue, more preferably 1 to 5 μg/ml of Factor VIIa (i.e. 3.4 to 16.7 UI/ml), and further preferably 2 to 4 μg/ml (i.e. 6.8 to 13.6 UI/ml).

When present, factor XIII is contained in a proportion of 2 UI/ml to 700 UI/ml, preferably 2 UI/ml to 10 UI/ml.

When present, the biological glue comprises 2 μmoles to 30 μmoles of the calcium ion source per ml of biological glue, more preferably 3 to 6 μmole/ml of the calcium ion source.

The Applicant has observed however that the best results in terms of the desired effects mentioned above can be obtained when the contents of the two coagulation factors, fibrinogen and FVIIa, and in particular their ratios, are specifically selected.

Therefore, in the biological glue of the invention the ratio of fibrinogen concentration to FVIIa concentration (the concentrations being expressed by weight per volume) is 60000:1 to 1000:1, more preferably 20000:1 to 1000:1 and further preferably 10000:1 to 1000:1.

According to one embodiment, the viscosity of the glue of the invention is higher than 1 mPa·s at 37° C. The viscosity of the biological glue of the invention is increased and corresponds to the viscosity obtained through the presence of at least one gelling agent in a content of 0.01% to 10% (by weight/volume, i.e. 0.01 to 10 gram(s)/100 ml of solution), more preferably 0.1% to 5%, or even 0.5 to 2%.

According to one embodiment, the glue comprises a source of calcium ions and its viscosity according to the invention is higher than 1 mPa·s at 37° C. The viscosity of the biological glue comprising a calcium ion source is increased and corresponds to the viscosity obtained through the presence of at least one gelling agent in a content of 0.01% to 10% (weight/volume, i.e. 0.01 to 10 gram(s)/100 ml of solution), more preferably 0.1% to 5%, or even 0.5 to 2%.

In the above-described cases, the gelling agent is a pharmaceutically acceptable gelling agent. In the present invention, a gelling agent refers to a gelling and/or thickening agent. Preferably, it is a derivative of a compound of natural origin, such as cellulose, preferably said gelling agent is hydroxypropyl cellulose. The gelling agent preferably has a molecular weight higher than 60 kDa, advantageously 500 to 2000 kDa, more advantageously 500 to 1000 kDa. For example, the gelling agent used may be Klucel MF (Ashland) of pharmaceutical grade. Other than cellulose derivatives, the gelling agent may also be selected from among a polymer of acrylic acid, chitosan, gelatine, alginates, carrageenans, and their respective derivatives, this list being non-limiting.

According to one embodiment of the invention, a mixture of several gelling agents can be used.

According to one embodiment of the invention, the glue comprising calcium ions comprises a mixture of several gelling agents.

The concentrations and activities are given per millilitre of final liquid solution of biological glue.

Said solution can particularly be obtained by reconstitution of the lyophilised components. As indicated above, these factors preferably contained in such concentrations ensure the desired functionalities of the present biological glue.

The liquid biological glue of the invention is stable since its components can be contained in the presence of a liquid medium for functional use thereof, even for at least 24 hours before use, without any risk of early triggering of the coagulation process. This stability of the glue is verified since no fibrin formation is observed during an incubation time of 24 hours at ambient temperature.

The biological glue of the invention fulfils its tissue repair function once applied to the wound or bleeding tissue, since thrombin is formed in situ by contact of the biological glue with the wound or bleeding tissue.

This in situ formation of thrombin is ensured by factor VIIa which is therefore placed in contact with all the plasmatic components allowing the triggering and occurrence of coagulation via the extrinsic or intrinsic pathway. As previously explained, its biological activity is dependent upon interaction with the tissue factor of endogenous phospholipid type. The activated factor VII/tissue factor complex in the presence of calcium ions converts the factor X contained in the plasma to activated factor X, which in turn converts prothrombin to thrombin, the enzyme responsible for clot formation by generating fibrin in the presence of fibrinogen.

Fibrin formation then takes place and the adhesive power of the biological glue obtained is reinforced, since fibrin is formed directly at the wound or tissue to be surgically repaired, where the phospholipid cell components are exposed.

Although fibrinogen is already naturally present in the blood, its addition further reinforces and accelerates the process of tissue joining and haemostasis via the biological glue of the invention, even when compared with prior art glues. This forms a decisive advantage of the present glue. Similarly, although calcium is already naturally present in the blood, the addition thereof further reinforces and accelerates the process of tissue joining and haemostasis via the biological glue of the invention, even when compared with prior art glues. In particular, the addition of calcium ions acts as co-factor of FVII and reinforces the activity of factor VIIa in situ. The adding of a gelling agent promotes contacting of the glue with the wound. This addition also enables limited dispersion of the glue at bleeding wounds, including major bleeding. The inventors have observed that the addition of gelling agent allows the stability of the glue to be increased and improves the coagulating effect.

The biological glue of the invention has numerous other advantages. The providing thereof avoids the disadvantages related to the handling and preparation of biological glues in the prior art, particularly in emergency situations, thereby improving tissue sealing capability in terms of dehiscence and absence of secondary bleeding at the scar in the progress of being formed.

For example, the glue of the invention avoids the formation of <<packets>> or clusters of fibrin seen in a wound due to inhomogeneous, independent setting of the glue probably subsequent to fibrin formation before contact with and adhesion to the wound. The scar formed also has a clean-cut line compared with the gluing obtained over the same time period when using a bi-compound biological glue based first on a mixture of fibrinogen containing Factor XIII, and secondly on a mixture of calcic thrombin, which does not achieve this effect.

It is simple to prepare and has increased stability over time since no fibrin formation is observed for 24 hours at ambient temperature. For example, it is stable in liquid form for at least 24 hours at ambient temperature, in particular for at least 2, 4 or 6 days.

As indicated above, the biological glue of the invention is ready to use and comprises a homogeneous mixture of all the components in liquid form. It may also be in frozen form, meaning that it can be stored for an extended time in this form for at least 2 years without any risk of fibrin formation which could be observed on thawing. The glue only needs to be brought back to ambient temperature so that it can be used.

The invention also concerns a thrombin-free, fibrinogen-based biological glue for therapeutic use comprising factor VIIa, such as previously defined, in lyophilised form and able to be stored for an extended time. It can be obtained by implementing known techniques for lyophilisation of glue in liquid form. The providing of this form of glue has the decisive advantage that it only necessitates mere reconstitution of the lyophilisate comprising the coagulation factors in a biologically compatible solvent or aqueous medium to obtain the stable liquid glue, this preparation being carried out ahead of planned use.

The invention also concerns a thrombin-free, fibrinogen-based biological glue for therapeutic use comprising factor VIIa and a source of calcium ions such as previously defined, in lyophilised form and able to be stored for an extended time. It can be obtained by implementing known techniques for lyophilisation of glue in liquid form. The providing of said form of the glue has the decisive advantage that it only requires mere reconstitution of the lyophilisate comprising the coagulation factors and calcium ion source in a biologically compatible solvent or aqueous medium to obtain the stable liquid glue, this preparation being carried out ahead of planned use.

According to another embodiment, the invention also concerns a thrombin-free, fibrinogen-based biological glue comprising factor VIIa and at least one gelling agent such as previously defined, in lyophilised form and able to be stored for an extended time. It can be obtained by implementing known techniques for lyophilisation of glue in liquid form. The providing of said form of the glue has the decisive advantage that it only requires mere reconstitution of the lyophilisate comprising the coagulation factors and gelling agent(s) in a biologically compatible solvent or aqueous medium to obtain the stable liquid glue, this preparation being carried out ahead of planned use. According to another more particular embodiment, the invention also concerns a thrombin-free, fibrinogen-based biological glue for therapeutic use comprising factor VIIa, a calcium ion source and at least one gelling agent such as previously defined, in lyophilised form and able to be stored for an extended time. It can be obtained by implementing known techniques for lyophilisation of glue in liquid form. The providing of said form of the glue has the decisive advantage that it only requires mere reconstitution of the lyophilisate comprising the coagulation factors, calcium ion source and gelling agent(s) in a biologically compatible solvent or aqueous medium to obtain the stable liquid glue, this preparation being carried out ahead of planned use.

In addition, said lyophilised glues are very easily stored at ambient temperature for at least 2 years without any risk of fibrin formation which could be observed after reconstitution. They can easily be transported to a site where a patient is in need of application of these glues.

### Kit

The invention also concerns a kit for preparing the biological glue of the invention comprising packaging means including a lyophilised fibrinogen pack, lyophilised factor FVIIa pack and an aqueous solvent.

The invention also concerns a kit for preparing the biological glue of the invention comprising packaging means including a lyophilised fibrinogen pack, lyophilised factor FVIIa pack, calcium ion source pack in powder form and an aqueous solvent.

Said kit, comprising different individual packs of each particular constituent component in dried form of the glue of the invention, is in fact an intermediate product of which the pack contents can easily be combined and dissolved in a biologically compatible solvent or aqueous medium, such as purified water for injection (PWI), to obtain the stable liquid biological glue of the invention, concentrated if necessary, and which can then be frozen. The advantage of providing said kit is the possibility of extended storage of the different packs for at least 2 years at ambient temperature, without observing any fibrin formation after reconstitution, and the advantage of obtaining the stable liquid biological glue by mere dissolution.

According to one embodiment of the invention, said kit further comprises at least one dried gelling agent component.

According to one embodiment of the invention, said kit further comprises a calcium ion source component in powder form.

According to one embodiment of the invention, said kit comprises at least one gelling agent component in dried form and a calcium ion source component in powder form.

The packaging means particularly form means allowing storage of the glue components, preparation thereof before use and the dispensing thereof. They further advantageously comprise at least one container for each component. Each container is intended to receive one of the components that is later dissolved in the aqueous solvent. The containers may be bottles in various materials of biologically compatible glass and polymer type.

Preferably, the packaging means may advantageously be a single container containing all the components. Said packaging has the advantage that mere dissolving of the whole directly provides the liquid glue ready to use.

Advantageously, the kit also comprises a device to dispense the liquid glue once prepared by dissolution of the above components in the aqueous solvent. Said device is a syringe for example of appropriate volume as a function of the dose of glue to be delivered, comprising a fine needle typically having a diameter of less than 2 mm, in particular less than 1 mm, or else a conventional catheter or spray device allowing the dispensing of a small volume of product onto a large surface, or a brush which can be used to spread a precise amount of reconstituted glue on the surface of the wound, or else a biocompatible and/or biodegradable fabric which holds the glue on the wound. The fabric may have been extemporaneously impregnated with the reconstituted glue or it may be fabricated in the presence of the glue. With this latter option, the fabric integrating the compounds of the biological glue is then ready for use and is deposited directly on the wound.

Preferably, the above kit comprises a multi-component pack comprising a mixture of said lyophilised factors. In one variant, this kit comprises a pack of lyophilised mixture of coagulation factors of the invention and a pack of calcium ion source in powder form which may also be a lyophilisate, these simply needing to be combined and mixed in an aqueous medium such as PWI water before use. As a variant, the kit may comprise a pack of lyophilised mixture of coagulation factors and a pack of calcium ion source, in the form of an aqueous solution. Advantageously, this kit also comprises at least one gelling agent in dried form, or in the form of an aqueous solution forming a bi-component pack in a mixture with the calcium ion source or forming a separate pack.

Preferably the kit of the invention is characterized by the fact that each pack of lyophilised fibrinogen or FVIIa, or the bi-component pack of the mixture of said lyophilised factors, comprises constituents of a pharmaceutically acceptable lyophilisation-stabilising formulation. The same applies to the lyophilised biological glue.

The different lyophilisates of the coagulation factors are obtained by lyophilisation of the concentrates or liquid solutions of the factors, or of each of these factors, using conventional techniques advantageously comprising for this purpose a lyophilisation-stabilising formulation.

The stabilising formulation, as needed, may comprise stabilising adjuvants known in the art.

The invention therefore also concerns the use of the kit of the invention to prepare a liquid biological glue by reconstitution of the components of the kit in a biologically compatible aqueous solvent, optionally followed by freezing.

### Therapeutic Applications

The invention also concerns a biological glue such as described above for use as medicament. In particular, the invention concerns a biological glue containing fibrinogen, activated Factor VII such as described above, for use as medicament. The invention also concerns a biological glue containing fibrinogen, activated factor VII and a source of calcium ions, such as described above, for use as medicament.

If the glue is stored in frozen form, it is sufficient to for the glue to be thawed before use.

If the glue is lyophilised alone or in contact with a fabric, reconstitution in a biologically compatible aqueous solvent allows use thereof to obtain the desired effects.

If the glue is in the form of a liquid formulation or impregnated fabric, it is ready for use.

The biological glue of the invention is therefore used to stimulate return to haemostasis and/or healing of damaged biological tissues such as the skin or any organ which may undergo surgical procedure (spleen, liver, lung, intestines, vessels etc.) which, as explained above, may have raw tissue or haemorrhagic wounds the bleeding of which may even be slight insofar as all the factors required for the coagulation cascade are present. For local applications of the glue to sites with bleeding, in particular raw tissues or haemorrhagic wounds, the glue is used alone without the addition of another coagulation factor e.g. without the addition of plasma.

The glue can be used in the presence of plasma to prepare a medicament, the latter advantageously being intended to treat damaged tissues e.g. to join these tissues, selected from the group formed by cartilage, collagen, bone and bone powder.

In addition, in the presence of plasma, it is also used to prepare a medicament intended to join biomaterials selected from the group formed by alginates or polylactic acid. The presence of plasma is therefore necessary in some cases for an exogenous supply of plasmatic factors to initiate the coagulation cascade, and preferably it is compatible or autologous plasma. This supply may be provided in particular for conventional surgical procedures, in stomatology or odontology.

The biological tissue may also derive from cultured and differentiated stem cells.

Although the glue of the invention is a medicament, in particular a dressing for local application i.e. for external use on a wound or other as described above, it is not excluded that the medicament may also be an adapted capsule whether or not gastro-resistant, in which the biological glue is in dried form and ingested to treat digestive bleeding.

The, at least, 24-hour stability of the glue of the invention enables it to remain fluid and hence able to be used to prepare a medicament intended to embolise the nourishing blood vessels of a tumour target. This is advantageously achieved via injection using a conventional catheter as far as these tumour targets to <<dry out>> the tumour.

The glue allows haemostasis to be obtained through an endoscopic system used in microsurgery (sampling, biopsies, polyp excision etc.).

The fluidity of the present glue, related in particular to the stability thereof, means that it can be used through a fine needle typically of diameter smaller than 1 mm, particularly smaller than 0.5 mm for application in surgery under microscope e.g. ophthalmic surgery.

The following Figures and Examples illustrate the invention without, however, limiting the scope thereof.

### FIGURES

FIG. 1 shows the influence of the gelling agent, its concentration and concentration of fibrinogen (Fbg) on the total formation time (CT+CFT) of the formed clot.

FIG. 2 shows the influence of the gelling agent, its concentration and concentration of fibrinogen on the firmness of the formed clot.

FIG. 3 shows the influence of the gelling agent, its concentration and concentration of fibrinogen on the efficacy of the formed clot (ratio of clot strength to rate of clot formation).

FIG. 4 gives studies on the stability of the glues in relation to total clot formation time (CT+CFT), without gelling agent at different concentrations of fibrinogen.

FIG. 5 gives studies on the stability of the glues in relation to clot firmness without gelling agent at different concentrations of fibrinogen.

FIG. 6 gives studies on the stability of the glues in relation to total clot formation time (CT+CFT) in the presence of 1% Klucel MF at different concentrations of fibrinogen.

FIG. 7 gives studies on the stability of the glues in relation to clot firmness in the presence of 1% Klucel MF at different concentrations of fibrinogen.

FIG. 8 illustrates the adhesive power at 15 minutes in the carotid region of eight glue compositions.

FIG. 9 illustrates the adhesive power at 15 minutes in the hepatic region of seven glue compositions.

FIG. 10 illustrates the adhesive power at 15 minutes in the spleen region of five glue compositions.

FIG. 11 illustrates hepatic bleeding in grams at 15 minutes after incisions and application of one of the eight tested glue compositions.

FIG. 12 illustrates spleen bleeding in grams at 15 minutes after incisions and application of one of the five tested glue compositions.

FIG. 13 illustrates kidney bleeding in grams at 15 minutes after incisions and application of one of the five tested glue compositions.

### EXAMPLES

### Example 1: Study on Type of Gelling Agent

Four compounds were tested for their glue gelling capability: Klucel HF (MW: 1 150 kDa), Klucel MF (MW: 850 kDa), PlasdoneC30 (MW: 58 kDa) and PlasdoneC17 (MW: 10 kDa).

Strong concentrations of the gelling agents PlasdoneC30 and PlasdoneC17 (concentrations higher than 30% in g/mL of solution) were needed to obtain satisfactory viscosity, contrary to Klucel HF and Klucel MF of which concentrations of 0.01 to 10% provided major thickening of the glue preparation. Therefore, the properties of the gelled glue were researched in the presence of Klucel HF and Klucel MF.

### Example 2: Influence of the Gelling Agent and Fibrinogen Concentration on Total Clot Formation Time

Gelling of the biological glue was measured by ROTEM. ROTEM is a measurement of thromboelastometry which systematically provides several parameters: coagulation time (CT) and clot formation time (CFT), two kinetic parameters characterizing the rapidity of coagulation of the solution. For greater simplicity, the value corresponding to the sum of these two parameters (CT+CFT) is given, corresponding to the total clot formation time. The system also provides a value reflecting maximum clot firmness (MCF in mm).

In brief, a solution of gelling agent was prepared by reconstituting the powder gelling agent in 25 mM HEPES buffer, 175 mM NaCl pH 7.4 for 16 h at ambient temperature. A 2× concentrated solution of gelling agent was thus prepared (1 or 2 g/100 ml). The next day, a volume of this gelling solution (or buffer for negative control) was added to a volume of 2× concentrated fibrinogen solution previously dialysed against the same HEPES buffer (fibrinogen solution of 6 to 90 mg/ml before dilution). FVIIa was then added in the desired concentration (2-5 μg/ml) in minimum volume (<1% of total volume). FVIIa was previously diluted against HEPES buffer. The experiment was initiated through the addition of 0.5 pM tissue factor, 4 μM phospholipids, 5% human plasma and 5 μM CaCl₂ in a small ROTEM dish (500 μl). Fibrin formation was recorded by increase in the density of the solution.

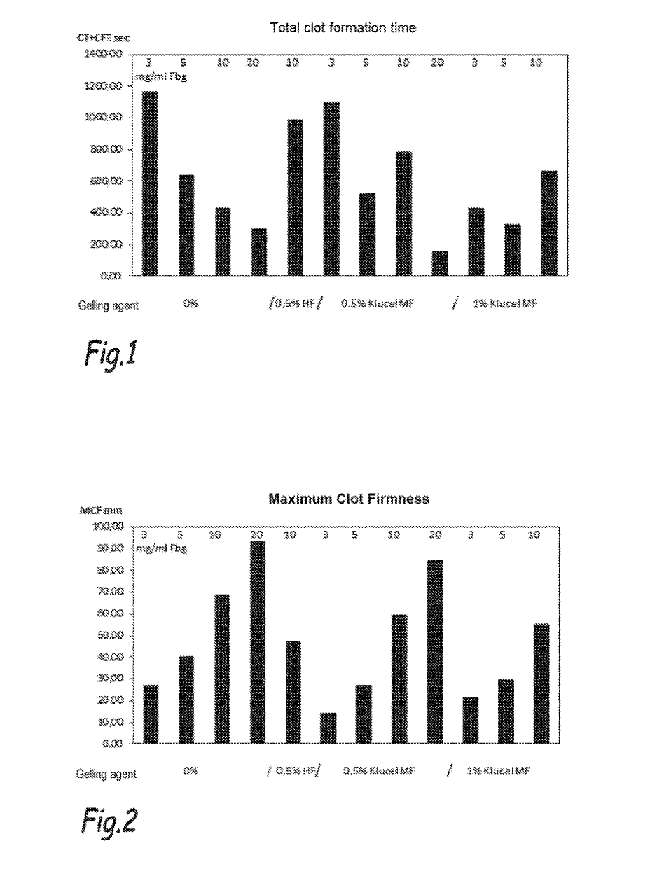

The sum of clot formation time and coagulation time was measured under each of the conditions (FIG. 1).

Without gelling agent, the increase in fibrinogen concentration leads to a regular decrease in clot onset time from 1164 to 300 s. In the presence of 0.5% Klucel HF, the times with 10 mg/ml fibrinogen remain higher than those obtained in the absence of gelling agent, this difference not being significant. In the presence of 0.5% or 1% Klucel MF, the clot formation times are similar to those without gelling agent, even lower with 20 mg/ml fibrinogen and 0.5% Klucel MF. As a result, the presence of Klucel MF or Klucel HF at between 0.5 and 1% does not significantly modify clot formation time.

### Example 3: Influence of Gelling Agent Concentration and Fibrinogen Concentration on Clot Strength

Clot firmness (MCF in mm) was measured by ROTEM as described in Example 2 (FIG. 2). Without gelling agent clot firmness increases regularly with fibrinogen concentration, increasing from 27 mm with 3 mg/ml fibrinogen to 93 mm with 20 mg/ml fibrinogen. The presence of 0.5% Klucel MF or Klucel HF does not significantly modify firmness, in particular on and after 10 mg/ml de fibrinogen.

### Example 4: Influence of Gelling Agent Concentration and Fibrinogen Concentration on the Efficacy of Clot Formation

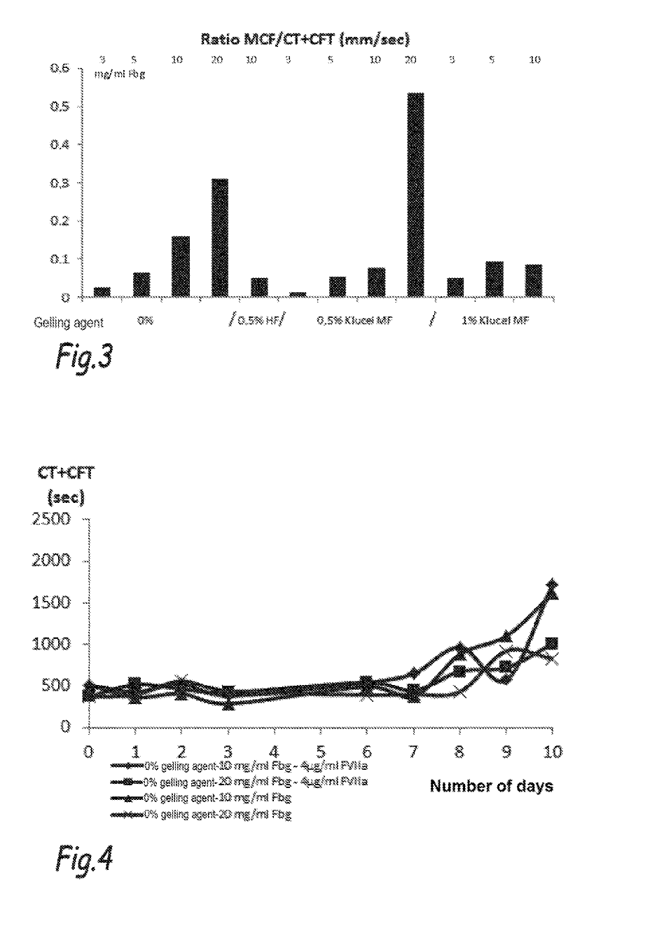

The ratio of clot firmness (in mm) to rate of clot formation (in sec) allows evaluation of the overall quality of clot formation in a single parameter. This ratio was calculated and is given as a function of different parameters of the study (FIG. 3). Without gelling agent, the ratio increases regularly as a function of fibrinogen concentration, reaching a maximum at 20 mg/ml (ratio=0.31). In the presence of 0.5% Klucel HF, this ratio is reduced with 10 mg/ml de fibrinogen. In the presence of 1% Klucel MF, the ratio is better with low concentrations of fibrinogen (3 and 5 mg/ml) than when not present. In the presence of 0.5% Klucel MF, the ratios are globally maintained relative to a solution without gelling agent. An optimum is seen in the region of 20 mg/ml fibrinogen.

These data show that the presence of the Klucel MF thickener in the solution of biological glue does not affect formation of the fibrin clot whilst increasing the viscosity of the solution.

### Example 5: Study on the Stability of Different Glues in Respect of Maximum Clot Firmness (MCF), Coagulation Time (CT) and Clot Formation Time (CFT)

The stability of several glue formulations was evaluated by ROTEM analysis. First, formulations of biological glues not containing any gelling agent were prepared and analysed. These were mixtures of 10 or 20 mg/ml fibrinogen incubated in the presence of FVIIa for 0 to 10 days or in which FVIIa was added extemporaneously. Polymerisation of the glue was triggered through the addition of an inducing mixture (0.5 pM tissue factor, 2 μM phospholipids, 3 mM calcium) in the presence of 5% human plasma.

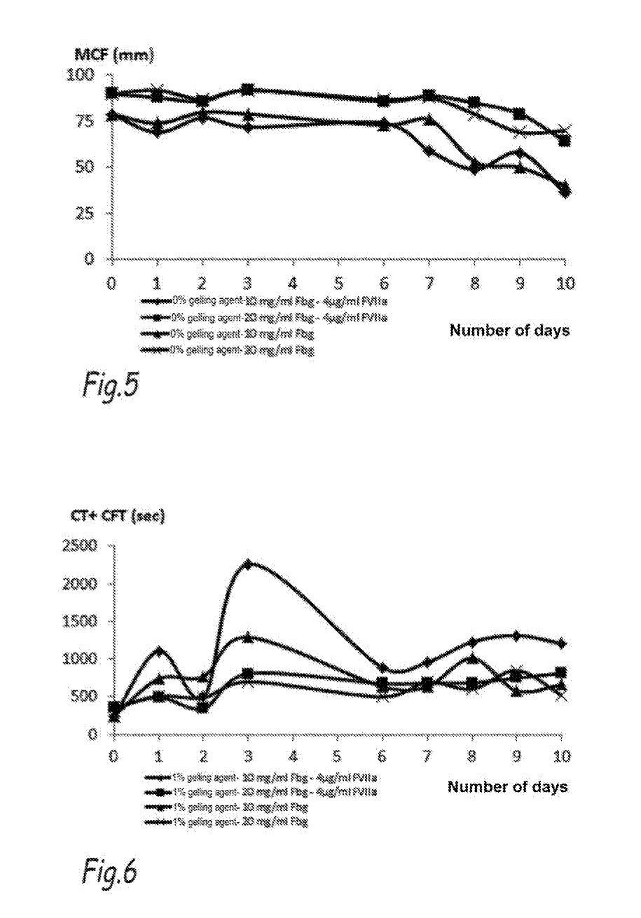

Incubation of the solutions for up to 7 days does not affect the rate of clot onset (CT+CFT) whether FVIIa was incubated in the presence of (Fbg-FVIIa) or added at the time of measurement (Fbg) (FIG. 4). After 7 days, the clot formation time increased regularly under all examined conditions. Clot firmness was optimal with 20 mg/ml fibrinogen, whether or not FVIIa was incubated in the presence of fibrinogen (FIG. 5, ▪ and x). There was no variation in firmness for the 7 first days after which it decreased regularly over time under all examined conditions. The clots formed from mixtures containing 20 mg/ml fibrinogen remained more resistant than those formed from 10 mg/ml solutions.

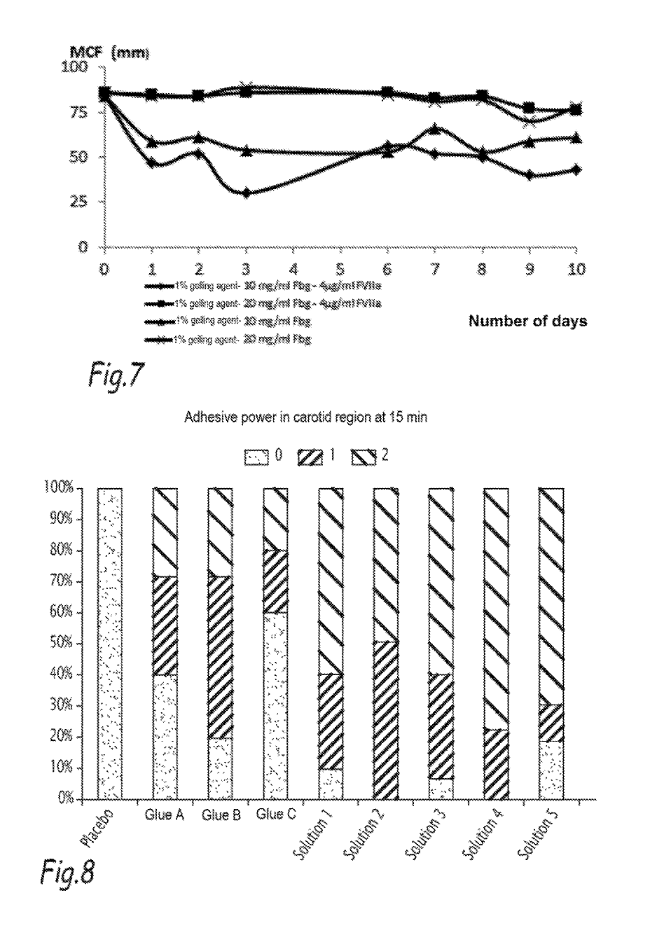

A similar experiment was repeated by adding 1% (1 g/100 mL solution) of Klucel MF gelling agent to the fibrinogen and incubating them together for different times (FIGS. 6 and 7). The presence of gelling agent did not cause any major modification in the quality of the formed clots. It even tended to limit loss of efficacy between days 7 and 10. As previously, the mixtures formed from 20 mg/ml fibrinogen gave clots that were more resistant and formed more rapidly.

To conclude, the preparation of biological glue can be incubated up to 7 days at 25° C. without losing or reducing its coagulation function. The addition of 1% Klucel MF improves the performance of the solution over longer time periods.

### Example 6: In Vivo Study on Eight Formulations of Biological Glues

In this study, several glue formulations were tested in a rabbit organ bleeding model. The eight tested formulations were denoted: Glue A, Glue B, Glue C (corresponding to 3 glue formulations with different fibrinogen concentrations) and Solution 1, Solution 2, Solution 3, Solution 4, Solution 5 (glue formulation with added gelling agent), all these solutions being compared with a placebo (buffer solution).
Glue A: 3 mg/ml Fibrinogen+2 μg/ml FVIIa
Glue B: 20 mg/ml Fibrinogen+2 μg/ml FVIIa
Glue C: 80 mg/ml Fibrinogen+2 μg/ml FVIIa
Solution 1: 3 mg/ml Fibrinogen+2 μg/ml FVIIa+1% (w/v) Klucel MF
Solution 2: 20 mg/ml Fibrinogen+2 μg/ml FVIIa+1% (w/v) Klucel MF
Solution 3: 10 mg/ml Fibrinogen (plasmatic origin)+2 μg/ml FVIIa+1% (w/v) Klucel MF
Solution 4: 10 mg/ml Fibrinogen (transgenic origin)+2 μg/ml FVIIa+1% (w/v) Klucel MF
Solution 5: 10 mg/ml Fibrinogen (transgenic origin)+2 μg/ml FVIIa+1% (w/v) Klucel MF+33 μg/ml FXIII

The primary evaluation criterion was organ bleeding, evaluated via the amount of blood lost after organ lesion. The secondary evaluation criterion was the adhesive power observed at the carotid vessels (without induced wound) and at the edges of the organ lesions, graded from 0 to 2 (0=no adhesive power, 1=low adhesion, 2=strong adhesion).

This blind, randomised, controlled study included a total of forty-eight female New Zealand rabbits weighing about 3 kg-4 kg, divided into 9 groups of 5 animals (with 3 pilot animals): Placebo group, glue A group, glue B group, glue C group, Solution 1 group, Solution 2 group, Solution 3 group, Solution 4 group and Solution 5 group. An organ bleeding model was applied to evaluate haemostatic capacity (capacity to limit bleeding) and adhesive power in rabbits with surgical wounds of organs. The entire experiment was conducted under general anaesthesia. The order of treatment (glue or placebo) was conducted by drawing lots.

After induced anaesthesia, the rabbits were tracheotomised and mechanically ventilated. First, the adhesive power was examined in the carotid region (without induced bleeding). At 5 and 15 minutes, the adhesive power was evaluated using a score of 0 to 2 (0=no adhesive power, 1=low adhesion, 2=strong adhesion).

Organ bleeding was then examined after median xyphopubic laparotomy and hepatic, splenic then renal lesions with blood collection over 15 minutes for each organ. The lost, collected blood was weighed. In more detail, first 6 standardised incisions were made perpendicular to the free edge of the liver and away from the vascular pedicle. The haemostatic product was applied to the lesions. Bleeding was evaluated after 15 minutes by weighing the compresses placed underneath and around the liver. The adhesive power at 5 and 15 minutes was graded between 0 and 2. Similarly, spleen bleeding was obtained after 4 incisions perpendicular to the convexity, then right and left renal bleeding after 3 transfixing wounds on each kidney. For each organ, after application of the haemostatic product, bleeding was measured at 15 minutes, and the adhesive power graded at 5 and 15 minutes for the liver and spleen.

On completion of the experiment, the animals were sacrificed by injection of a lethal dose of pentobarbital.

Three pilot animals allowed adaption and standardisation of the type and number of lesions for each organ.

TABLE 1
Study on the adhesive power of different formulations

| | Placebo | Glue A | Glue B | Glue C |
|---|---|---|---|---|
| Carotid | | | | |
| score 5 min* | 0/20 | 0/20 | 10/20 | 3/20 |
| median 5 min | 0 (0-0) | 0 (0-0) | 1 (0-2) | 0 (0-2) |
| score 15 min* | 0/20 | 9/20 | 11/20 | 6/20 |
| median 15 min | 0 (0-0) | 1 (0-2) | 1 (0-2) | 0 (0-2) |
| p score vs placebo | | 0.005 | <0.001 | 0.03 |
| Liver | | | | |
| score 5 min ** | 0/10 | 3/10 | 4/10 | 3/10 |
| median 5 min | 0 (0-0) | 1 (0-1) | 1 (0-1) | 0 (0-2) |
| score 15 min ** | 0/10 | 3/10 | 6/10 | 4/10 |
| median 15 min | 0 (0-0) | 1 (0-1) | 1 (0-2) | 1 (0-2) |
| p score vs placebo | | 0.05 | 0.018 | NS _{(0.053)} |
| Spleen | | | | |
| score 5 min ** | 0/10 | 1/10 | 3/10 | 1/10 |
| median 5 min | 0 (0-0) | 0 (0-1) | 0 (0-2) | 0 (0-1) |
| score 15 min ** | 0/10 | 3/10 | 5/10 | 1/10 |
| median 15 min | 0 (0-0) | 0 (0-2) | 1 (0-2) | 0 (0-1) |
| p score vs placebo | | NS | NS _{(0.053)} | NS |
| N° of animals | 5 | 5 | 5 | 5 |

Glues A et B have stronger adhesive power than the placebo in the carotid and liver regions. Glue C, having the highest fibrinogen content, only has a significantly stronger adhesive power than the placebo in the carotid region. Therefore, the use of glues having fibrinogen concentrations lower than 60 mg/ml generally allows an increase in the adhesive power of the glue.

TABLE 1bis
Study on the adhesive power of different formulations

| | Placebo | Solution 1 | Solution 2 | Solution 3 | Solution 4 | Solution 5 |
|---|---|---|---|---|---|---|
| Carotid | | | | | | |
| score 5 min* | 0/20 | 8/20 | 10/20 | 26/52 | 20/52 | 7/20 |
| median 5 min | 0 (0-0) | 1 (0-2) | 1 (0-2) | 1 (0-2) | 1 (0-2) | 1 (0-2) |
| score 15 min* | 0/20 | 15/20 | 15/20 | 40/52 | 46/52 | 15/20 |
| median 15 min | 0 (0-0) | 2 (0-2) | 1.5 (1-2) | 2 (0-2) | 2 (1-2) | 2 (0-2) |
| p score vs placebo | | <0.001 | <0.001 | <0.001 | <0.001 | |
| Liver | | | | | | |
| score 5 min ** | 0/10 | 8/10 | 3/10 | 11/26 | 13/26 | |
| median 5 min | 0 (0-0) | 2 (0-2) | 1 (0-1) | 1 (0-2) | 1 (0-2) | |
| score 15 min ** | 0/10 | 8/10 | 7/10 | 12/26 | 15/26 | |
| median 15 min | 0 (0-0) | 2 (1-2) | 1 (1-2) | 1 (0-2) | 1 (0-2) | |
| p score vs placebo | | 0.005 | 0.005 | 0.015 | 0.004 | |
| Spleen | | | | | | |
| score 5 min ** | 0/10 | 1/10 | 2/10 | | | |
| median 5 min | 0 (0-0) | 0 (0-1) | 0 (0-2) | | | |
| score 15 min ** | 0/10 | 4/10 | 3/10 | | | |
| median 15 min | 0 (0-0) | 1 (0-1) | 1 (0-1) | | | |
| p score vs placebo | | 0.014 | 0.05 | | | |
| No of animals | 5 | 5 | 5 | 13 | 13 | 5 |

*sum of scores (of both carotids), at 5 and 15 minutes after application
** sum of scores, at 5 and 15 minutes after application
median (minimum-maximum)
NS: non-significant

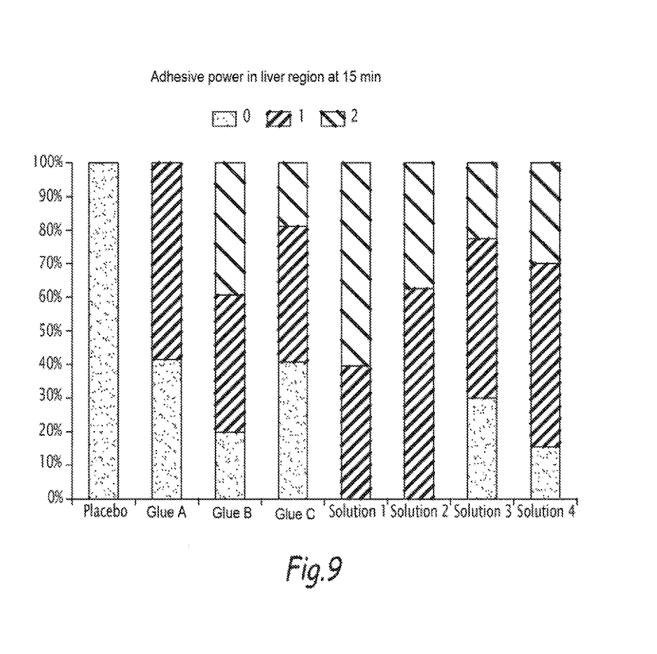

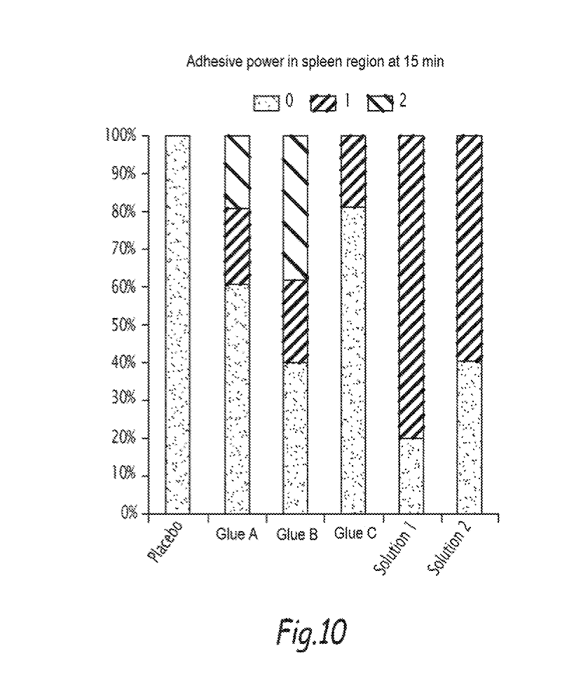

Solutions 1 to 5 have stronger adhesive power than the placebo on the tested regions (FIGS. 8, 9, 10). In addition, the use of gelling agent most advantageously allows an increase in the adhesive power of the glue (comparison of glues A and B and of Solutions 1 and 2).

TABLE 2
Study on blood loss

| | Placebo | Glue A | Glue B | Glue C |
|---|---|---|---|---|
| Liver | | | | |
| Loss in g | 4.40 | 1 | 2.11 | 2.91 |
| | (2.73-8.57) | (0.62-4.62) | (1.38-6.60) | (1.38-5.45) |
| p vs placebo | | 0.028 | NS | NS |
| Spleen | | | | |
| Loss in g | 19.20 | 11.47 | 11.41 | 14.39 |
| | (5.86-23.43) | (5.11-28.15) | (6.47-21.47) | (4.86-27.98) |
| p vs placebo | | NS | NS | NS |
| Kidneys | | | | |
| Loss in g | 3.22 | 4.21 | 3.14 | 4.0 |
| | (1.67-4.09) | (3.75-9.79) | (2.36-8.32) | (1.9-9.0) |
| p vs placebo | | 0.028 | NS | NS |
| N° of animals | 5 | 5 | 5 | 5 |

TABLE 2bis
Study on blood loss

| | Placebo | Solution 1 | Solution 2 | Solution 3 | Solution 4 | Solution 5 |
|---|---|---|---|---|---|---|
| Liver | | | | | | |
| Loss in g | 4.40 | 1.99 | 2.83 | 1.08 | 0.94 | 0.83 |
| | (2.73-8.57) | (1.05-7.60) | (0.90-6.64) | (0.53-2.18) | (0.46-5.81) | (0.53-2.18) |
| p vs placebo | | NS (0.076) | NS | 0.001 | 0.005 | |
| Spleen | | | | | | |
| Loss in g | 19.20 | 7.8 | 6.21 | | | |
| | (5.86-23.43) | (5.58-15.30) | (4.13-12.65) | | | |
| p vs placebo | | NS | NS | | | |
| Kidneys | | | | | | |
| Loss in g | 3.22 | 1.29 | 0.74 | | | |
| | (1.67-4.09) | (1.28-4.06) | (0.62-2.33) | | | |
| p vs placebo | | NS (0.075) | 0.016 | | | |
| No of animals | 5 | 5 | 5 | 13 | 13 | 5 |

Blood losses are expressed as a median (minimum-maximum).

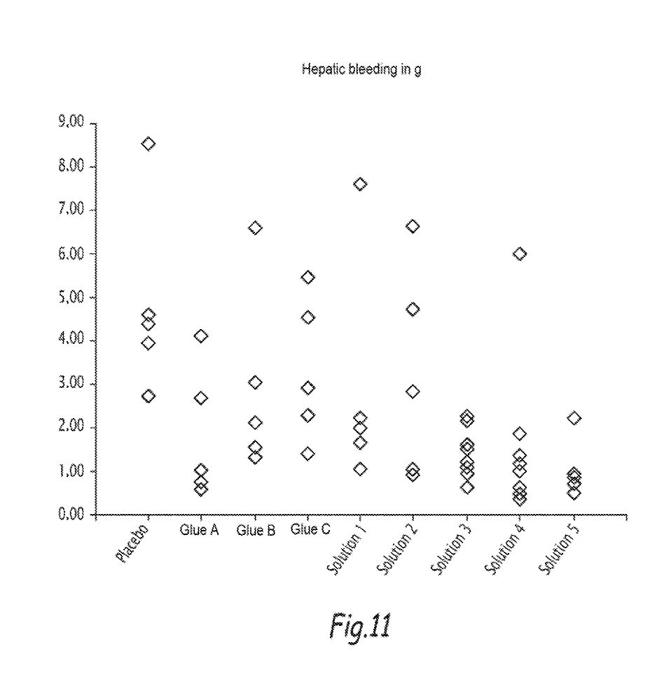

Hepatic bleeding in the <<Glue A>> group is statistically lower than in the placebo group. On account of an extreme value in the Solution 1 group, there is no significant difference between the a <<Solution 1>> group and placebo group (FIG. 11). However, a significant trend is observed towards a reduction in hepatic bleeding in the Solution 3 and 4 groups.

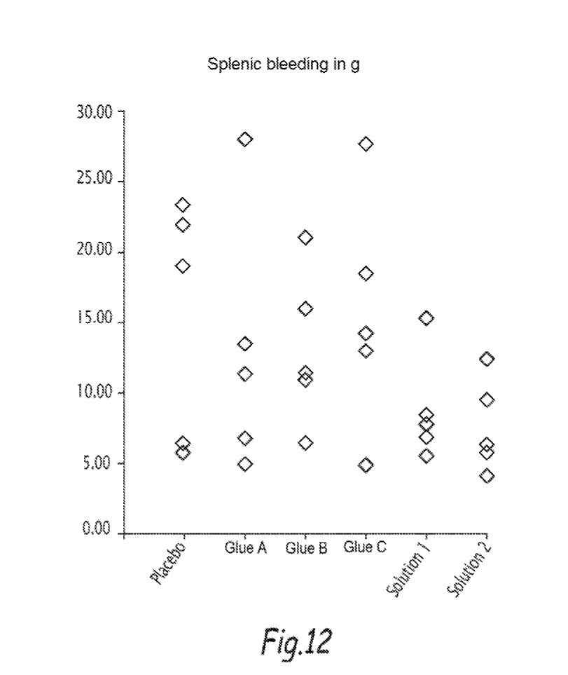

Also, there is no significant difference between the different groups regarding spleen bleeding on account of a wide dispersion of values (FIG. 12). However, a non-significant trend is observed towards reduced spleen and liver bleeding in the Solution 1 and 2 groups.

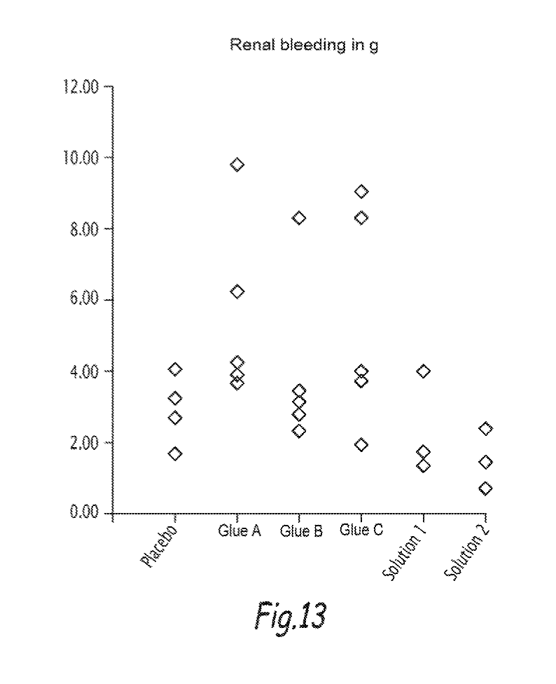

It appears that Solutions 1 and 2 allow a reduction in renal bleeding, although for Solution 1 this effect is not significant (FIG. 13).

In general, the use of fibrinogen concentrations lower than 60 mg/ml appears to be preferable to limit bleeding. The use of gelling agent in the glue formulation may also allow a reduction in bleeding, in particular in the spleen and kidney regions.

### Example 7: In Vivo Study on Healing Property

Two glues were tested in pigs after skin sampling. The grafts were bandaged in algostéril® alone or in the presence of glues comprising 45 mg/ml fibrinogen.

Four skin samples (7 cm×7 cm×4, i.e. about 15% of body surface) were taken using a dermatome from the back of Large White Landras pigs under general anaesthesia. Glue 1 comprised 20 mg/mL fibrinogen, 4 μg/mL FVIIa and 1% Klucel MF, Glue 2 comprised 45 mg/mL fibrinogen, 4 μg/mL FVIIa and 1% Klucel MF. Both glues were tested in association with a Jelonet® membrane (Smith & Nephew, London, UK) and the clinical progress of healing was compared with application of algostéril® at D2, 5 and 14.

Algostéril® containing calcium alginate is a haemostatic dressing conventionally employed in surgery for wound management. The Jelonet® was only used here as substrate for the glue of the invention, since it is advantageously employed as non-adhesive dressing and does not intrinsically contain any healing active ingredient.

<<100%>> healing denotes complete healing. The percentage represents the healed surface of the total lesioned surface area (49 cm²).

Table 3 gives the results obtained.

TABLE 3
Progress of healing as a function of treatment

| | Glue 1 (n = 4) | Glue 2 (n = 4) | Algostéril (n = 3) |
|---|---|---|---|
| Day 2 | 0% | 0% | 3% |
| Day 5 | 82% | 52.5% | 5% |
| Day 14 | 100% | 82.5% | 70% |

These results show that the glues used allowed a very signification acceleration of the healing process compared with the standard dressing method.

1-26. (canceled)

27. Thrombin-free, liquid biological glue for therapeutic use, comprising fibrinogen and factor VIIa, wherein the ratio of fibrinogen concentration to FVIIa concentration is 20000:1 to 1000:1, with the concentrations being expressed in weight per volume, and wherein the fibrinogen concentration is lower than 60 mg/ml.

28. The biological glue according to claim 27, having a fibrinogen content of 0.1 mg to 45 mg per ml of biological glue.

29. The biological glue according to claim 27, having a FVIIa content of 0.1 μg to 10 μg per ml of biological glue.

30. The biological glue according to claim 27, comprising a source of calcium ions.

31. The biological glue according to claim 30, comprising 2 μmoles to 30 μmoles of the calcium ion source per ml of biological glue.

32. The biological glue according to claim 27, further comprising at least one gelling agent.

33. A therapeutic method comprising administering a thrombin-free, liquid biological glue comprising fibrinogen, factor VIIa and at least one gelling agent.

34. The therapeutic method according to claim 33, wherein the biological glue has a fibrinogen content lower than 60 mg/ml.

35. The therapeutic method according to claim 33, wherein the biological glue has a FVIIa content of 0.1 μg to 10 μg per ml of biological glue.

36. The therapeutic method according to claim 33 wherein the biological glue comprises a source of calcium ions.

37. The biological glue according to claim 32, wherein said at least one gelling agent is a cellulose derivative.

38. The biological glue according to claim 32, wherein said at least one gelling agent derived from cellulose is hydroxypropyl cellulose.

39. The biological glue according to claim 32, wherein said at least one gelling agent is contained in a proportion of 0.01 to 10% by weight/volume (i.e. 0.01 to 10 gram(s)/100 ml of solution).

40. The biological glue according to claim 32, wherein said at least one gelling agent has a molecular weight higher than 60 kDa.

41. The biological glue according to claim 27, further comprising factor XIII.

42. Kit for preparing the biological glue according to claim 27, comprising packaging means including a lyophilised fibrinogen factor pack, lyophilised factor FVIIa pack, an aqueous solvent and optionally a gelling agent(s) pack in powder form and calcium ion source pack in powder form.

43. A method to prepare a biological glue such as defined claim 27 comprising: providing a kit for preparing the biological glue, the kit comprising packaging means including a lyophilised fibrinogen factor pack, lyophilised factor FVIIa pack, an aqueous solvent and optionally a gelling agent(s) pack in powder form and calcium ion source pack in powder form; and reconstituting the components of the kit in a biologically compatible aqueous solvent.

44. A medicament comprising the biological glue such as defined according to claim 27.

45. A method to stimulate return to haemostasis and/or healing of lesioned biological tissues comprising administering the biological glue according to claim 27.

46. A method to stimulate return to haemostasis and/or healing of lesioned biological tissues comprising administering the biological glue according to claim 27 which is in contact with a fabric or membrane (biocompatible and/or biodegradable).
